(19) United States
(12) Patent Application Publication
(10) Pub. No.: US 2018/0085056 A1
(43) Pub. Date: Mar. 29, 2018

(54) Sensory Apparatus for Detection of Peripheral Neuropathy

(71) Applicant: SEATTLE GENETICS, INC., Bothell, WA (US)

(72) Inventor: Danya Joy Youngblood, Mukilteo, WA (US)

(73) Assignee: SEATTLE GENETICS, INC., Bothell, WA (US)

(21) Appl. No.: 15/713,025

(22) Filed: Sep. 22, 2017

Related U.S. Application Data
(60) Provisional application No. 62/398,763, filed on Sep. 23, 2016.

(51) Int. Cl. A61B 5/00

(52) U.S. Cl. CPC A61B 5/4827; A44B 15/00

(57) ABSTRACT

A texture ring system is configured to aid individuals in detecting the symptoms of peripheral neuropathy. The system includes planar components that each have a differently textured surface, and a connector component that secures the planar components together in a circular ring.

### CROSS REFERENCE TO RELATED APPLICATIONS

This application claims the benefit under 35 U.S.C. 119(e) of U.S. Provisional Patent Application No. 62/398,763 filed Sep. 23, 2016, which is incorporated by reference herein in its entirety.

### FIELD OF THE INVENTION

This disclosure relates to a sensory apparatus, and more specifically, to differently textured surfaces attached to a ring for use in the detection of peripheral neuropathy.

### BACKGROUND OF THE INVENTION

Peripheral neuropathy is a condition impairing the peripheral nervous system. This condition can cause weakness, numbness, and pain in affected individuals. Generally, peripheral neuropathy prevents these individuals from feeling normal sensations in their hands and feet (or other parts of the body). The condition can be caused by various factors: genetic diseases, vitamin deficiency, and side effects from chemotherapy, among others.

Around 20 million people in the United States are affected by peripheral neuropathy. While doctors are able to detect signs of peripheral neuropathy in patients, additional methods are needed for patients themselves to have the ability to recognize symptoms of the condition. The present invention solves this and other problems.

### BRIEF SUMMARY OF THE INVENTION

Described herein is an apparatus for the detection of peripheral neuropathy. In an embodiment, this apparatus is a texture ring system, or sensory ring system.

In one aspect this disclosure provides a texture ring system comprising a plurality of planar components, each planar component comprising a textured surface that is tactually distinct from the textured surface of another of the plurality of planar components. The texture ring system also comprises a connector component configured to secure the plurality of planar components, the connector component comprising a circular ring.

In an embodiment, one of the textured surfaces of the texture ring system comprises ribbed lines, a rock salt pattern, a fishnet pattern, a concave bump pattern, a convex bump pattern, a checkered pattern, or a sandpaper pattern. In an embodiment, the planar component of the texture ring system comprises an oblong shape, a circular shape, or a rectangular shape. In an embodiment, the planar component the texture ring system comprises a circular inner component, the circular inner component comprising the textured surface.

In an embodiment, the planar component of the texture ring system comprises an opening, the opening configured to be threaded through the circular ring for securing the planar component to the connector component. In an embodiment, each planar component of the texture ring system is between 2 and 3 inches in length and between 1 and 2 inches in width. In an embodiment, each planar component of the texture ring system is composed of plastic. In an embodiment, the connector component of the texture ring system is composed of metal.

### BRIEF DESCRIPTION OF THE DRAWINGS

FIG. 1 illustrates a perspective view of the texture ring system, according to an embodiment.

FIG. 2 illustrates various planar components of the texture ring system, each having a different textured surface, according to an embodiment.

### DETAILED DESCRIPTION

The figures and following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system for purposes of illustration only. One skilled in the art will readily recognize from the following description that alternative embodiments of the structures and methods illustrated herein may be employed without departing from the principles described herein.

Described herein is a sensory apparatus for the detection of peripheral neuropathy. The apparatus provides a plurality of differently textured surfaces for a user to feel with a finger or thumb. A user who cannot perceive the distinction between one textured surface and another may be exhibiting a sign of peripheral neuropathy. In an embodiment, the sensory apparatus is a texture ring system, or sensory ring system. The texture ring system includes a plurality of planar components, each having a differently textured surface. In other embodiments, the sensory apparatus comprises a three-dimensional shape with a plurality of sides, each side having a differently textured surface; a single piece of paper or other component having a plurality of different textures on its surface; a plurality of pieces of paper or other components, each piece having a differently textured surface; or any apparatus having differently textured surfaces.

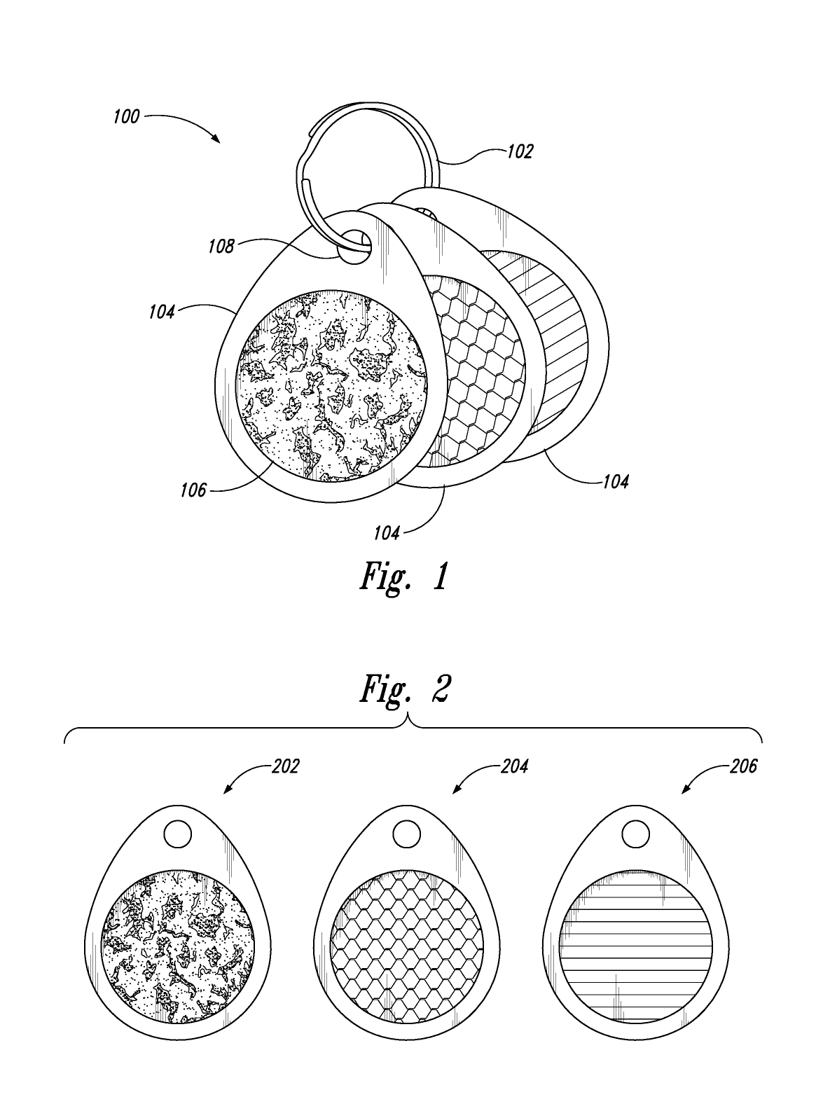

FIG. 1 illustrates a sensory apparatus that is a texture ring system 100. According to the embodiment illustrated in FIG. 1, the texture ring system 100 includes a connector component 102 and three flat planar components 104. In other embodiments, the texture ring system 100 comprises two, four, five, or six or more planar components 104. In an embodiment, the planar component 104 has an oblong and rounded egg shape. In other embodiments, the planar component 104 can be circular, oval, rectangular, or any other shape. The planar component 104 can be any size. For example, a planar component 104 can have a length of 1 inch, 2 inches, 3 inches, 4 inches, 5 inches, or longer, and can have a width of 1 inch, 2 inches, 3 inches, 4 inches, 5 inches, or longer. In an embodiment, the planar component 104 has a length between 2 and 3 inches and a width between 1 and 2 inches. In another embodiment, the planar component 104 has a length of 2.25 inches and a width of 1.75 inches. The planar component 104 may be composed of any material, such as plastic, metal, silicone, or rubber. In an embodiment, the planar component 104 is composed of plastic.

The planar component 104 has a textured surface 106. The textured surface 106 can cover the entirety of the planar component 104, or it can be present on a portion of the planar component 104. In the embodiment illustrated in FIG. 1, the textured surface 106 is present in a circular inner component of the planar component 104. In an embodiment, the circular inner component is an opening within the planar component 104, and the textured surface 106 is inlaid into the planar component 104 such that the textured surface 106 is tactual within the opening. In a further embodiment, the circular inner component is 1.5 inches in diameter. The textured surface 106 can have one of any number of textures. FIG. 1 shows three planar components with textured surfaces exhibiting (from left to right) a rock salt pattern, a fishnet pattern, and ribbed lines. The textured surface 106 may be composed of any material, such as plastic, metal, silicone, or rubber. In an embodiment, textured surface 106 is composed of plastic.

In an embodiment, the planar component 104 also has an opening 108 near an edge of the component. The opening can be a hole, a slit, or any other type of opening. This opening 108 is configured to allow a connector component 102 of the texture ring system 100 to thread through the opening 108. In an embodiment, the connector component 102 is a circular ring, such as a keyring, that is used to secure the planar components 104. The connector component 102 could also be a differently shaped band, such as a square or a flexible loop, or any other component that is configured to secure the planar components 104. The connector component 102 can be composed of any material, such as plastic, metal, silicone, or rubber. In an embodiment, the connector component 102 is composed of metal.

FIG. 2 more clearly illustrates the textured surfaces 106 of the planar components 104 illustrated in FIG. 1. From left to right, the textured surfaces 106 exhibit a rock salt pattern 202, a fishnet pattern 204, and ribbed lines 206. In other embodiments, the textured surface 106 can be a concave bump pattern, a convex bump pattern, a checkered pattern, a sandpaper pattern, or any pattern that a normal individual can perceive by touch. The textured surfaces 106 can also exhibit varying degrees of a texture, such as roughness, softness, fuzziness, or sharpness. In general, each of the planar components 104 of a texture ring system 100 has a tactually different textured surface 106 from the textured surface 106 of the other planar components 104 in the texture ring system 100. A textured surface 106 is tactually different from another if a person without peripheral neuropathy can distinguish between the two textured surfaces 106.

Throughout this specification, the terms “comprises,” “comprising,” “includes,” “including,” “has,” “having” or any other variation thereof, are intended to cover a non-exclusive inclusion. For example, a method, article, apparatus, or device that comprises a list of elements is not necessarily limited to only those elements but may include other elements not expressly listed or inherent to such method, article, apparatus, or device.

As used herein any reference to “one embodiment” or “an embodiment” means that a particular element, feature, structure, or characteristic described in connection with the embodiment is included in at least one embodiment. The appearances of the phrase “in one embodiment” are not necessarily all referring to the same embodiment.

Upon reading this disclosure, those of skill in the art will appreciate still additional alternative structural and functional designs as disclosed from the principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A texture ring system comprising:
a plurality of planar components, each planar component comprising a textured surface that is tactually distinct from the textured surface of another of the plurality of planar components; and
a connector component configured to secure the plurality of planar components, the connector component comprising a circular ring.

2. The texture ring system of claim 1, one of the textured surfaces comprising ribbed lines, a rock salt pattern, a fishnet pattern, a concave bump pattern, a convex bump pattern, a checkered pattern, or a sandpaper pattern.

3. The texture ring system of claim 1, the planar component comprising an oblong shape, a circular shape, or a rectangular shape.

4. The texture ring system of claim 1, the planar component further comprising a circular inner component, the circular inner component comprising the textured surface.

5. The texture ring system of claim 1, the planar component further comprising an opening, the opening configured to be threaded through the circular ring for securing the planar component to the connector component.

6. The texture ring system of claim 1, wherein each planar component is between 2 and 3 inches in length and between 1 and 2 inches in width.

7. The texture ring system of claim 1, wherein each planar component is composed of plastic.

8. The texture ring system of claim 1, wherein the connector component is composed of metal.

9. A texture ring system comprising:
three plastic planar components, each planar component comprising an oblong shape, and each planar component comprising a circular inner component having a textured surface that is tactually distinct from the textured surface of another of the circular inner components; and
a connector component configured to secure the three planar components, the connector component comprising a circular metal ring.

10. A texture ring system comprising:
three plastic planar components, each planar component comprising an oblong shape, and each planar component comprising a circular inner component having a textured surface, wherein a first textured surface comprises a rock salt pattern, a second textured surface comprises a fishnet pattern, and a third textured surface comprises ribbed lines; and
a connector component configured to secure the three planar components, the connector component comprising a circular metal ring.
